(19) United States
(12) Patent Application Publication
(10) Pub. No.: US 2015/0335432 A1
(43) Pub. Date: Nov. 26, 2015

(54) BIOCOMPATIBLE CONTAINMENT MEMBER FOR BONE AUGMENTATION SURGERY MADE OF PROCESSED NATURAL MEMBRANE FROM AN ANIMAL DONOR

(71) Applicant: OSSEOUS TECHNOLOGIES OF AMERICA, Newport Beach, CA (US)

(72) Inventor: David CHEUNG, Arcadia, CA (US)

(21) Appl. No.: 14/377,768

(22) PCT Filed: Feb. 11, 2013
(86) PCT No.: PCT/US13/25557

§ 371 Date: Aug. 8, 2014

Related U.S. Application Data
(60) Provisional application No. 61/597,131, filed on Feb. 9, 2012.

(51) Int. Cl. A61F 2/28; A61L 27/36; A61L 27/12; A61L 27/58

(52) U.S. Cl. CPC A61F 2/2846; A61L 27/58; A61L 27/3687; A61L 27/365; A61L 27/3625; A61L 27/12; A61L 2430/02; A61L 2430/40

(57) ABSTRACT

A surgically implantable containment member for maintaining a bone augmentation material in a desired location and/or configuration following implantation in a human or other mammalian patient, in which the containment member is made of a natural membrane, such as pericardium, isolated from an animal donor and processed to avoid inflammation or tissue rejection, and a method of bone augmentation using such a containment member. In a particularly preferred embodiment, the containment member has a window which rapidly dissipates upon exposure to bodily fluids after implantation to expose bone augmentation material contained within the containment member to an adjacent bone to be augmented.

### BACKGROUND OF THE INVENTION

Bone is the body's primarily structural tissue; consequently it can fracture and biomechanically fail. Fortunately, it has a remarkable ability to regenerate because bone tissue contains stem cells which are stimulated to form new bone within bone tissue and adjacent to the existing bone. Boney defects regenerate from stem cells residing in viable bone, stimulated by signally proteins, and multiplying on existing cells or on an extracellular matrix (i.e., trellis). Like all tissues, bone requires support via the vascular system to supply nutrients and cells, and to remove waste. Bone will not regenerate without prompt regeneration of new blood vessels (i.e., neovascularization), typically with the first days and weeks of the regenerative cascade.

After tooth loss, the adjacent jawbone (maxilla or mandible) frequently resorbs or atrophies. This may cause problems when it is desired to replace a missing tooth with a dental implant because the required depth of bone needed to adequately support the implant may not be present. Thus, prior to implanting a dental implant, it is often necessary for the oral surgeon to regenerate the adjacent bone to at least the minimum depth to provide adequate osteointegration of the dental implant. A common procedure for this purpose is alveolar ridge augmentation.

Various attempts have been made in the past to stimulate or augment bone regeneration by introducing a bone regenerating material proximate a deteriorated bone structure. Examples of such materials made of reconstituted collagen fibers are disclosed in US patent publication nos. US 2007/0042326 and US 2011/0035024, the entire disclosures of each of which are incorporated herein by reference. Reconstituted collagen containment members, including capsules, wedges, curves, colladiscs and gullwings are typically made from purified bovine Achilles tendons. The tendons are cut and homogenized prior to pepsin digestion. At the end of the digestion, pepsin as well as all digested non-collagen materials are removed by washing with multiple steps of acid and neutral salt washes while keeping the collagen fibers intact. The resulting purified collagen is further washed into an alcohol solution and the final preparation is suspended into a slurry in alcohol solution. The resulting collagen slurry is cast into molds with the desired shapes and sizes and then dried to the desired thickness and physical consistency through freeze drying as well as drying under different temperatures. The final product is trimmed to specified dimensions, packaged and sterilized by gamma-irradiation. Such devices have found wide commercial acceptance, but because they are made of a reconstituted material, there are limits to their flexibility when dry and to their tensile strength when exposed to bodily fluids.

Other graft containment member products are synthetic materials or polymers, some resorbable and others not resorbable, which in practical application leave much to be desired.

### SUMMARY OF THE INVENTION

The presently claimed invention proceeds along an entirely different approach starting from a natural tissue membrane isolated from an animal donor to provide a biocompatible containment member for oral surgery or orthopedic surgery applications with enhanced versatility and usefulness.

In accordance with the present invention, a natural connective tissue membrane isolated from an animal donor source is processed such that the collagen fibers may be implanted in a recipient without an immune and inflammatory rejection. The collagenous connective tissue is subjected to a process that permits the collagen fibers to remain structurally intact and also enables the tissue to be implanted in a human recipient without an immune and inflammatory rejection.

After removal from the donor, the collagenous connective tissue is trimmed in saline and thereafter the collagen fibers are stabilized in a cold stabilizing solution having a temperature range of 4 to 10° C. The cold stabilizing solution may be a saline solution where the collagen fibers are soaked preferably for a period less than 48-hours, or an alcohol/water solution where the soaking time preferably does not exceed 30 days.

After the tissue is stabilized, it is submerged and soaked in a solution comprised of polyglycol, a salt, a phosphate buffer, and an oxidizing agent. The concentration of the polyglycol is in the range of 1% to 15% and its molecular weight may be in the range of 2,000 Daltons to 20,000 Daltons; the salt concentration may be in the range of 2.5M 4.5M (moles per liter of solution). The phosphate buffer is selected from the group consisting of sodium phosphate and potassium phosphate. It is preferable that the buffer have a concentration of 0.05M with a pH of 7.4. However, the pH may have a range between 6.5 and 7.8 and the concentration may range from 0.02 to 0.1M. The oxidizing agent preferably is hydrogen peroxide having a concentration in the range of 0.1% to 2%. However, ozone may also be used as an oxidizing agent in an alternative variant in a concentration range of 1 500 ppm, preferably in the range of 20 40 ppm.

Following soaking in the first or masking solution, the tissue is washed in a second solution comprised of alcohol and water where the alcohol may be selected from the group consisting of ethanol, iso-propanol, n-propanol, and combinations of different alcohols.

After washing the residue remaining on the tissue from soaking in the polyglycol, salt, phosphate buffer and oxidizing agent solution, the tissue is further soaked in a third solution containing alcohol, water and an anti-inflammatory agent selected from the group consisting of indomethacin, ibuprofin, aspirin, choline salicylate, difunisal, magnesium salicylate, magnesium choline salicylate, salsalate, flurbiprofen, fenoprofen, ketoprofen, naprosen, naproxen sodium, oxaprozin, diclofenac sodium, diclofenac misoprostol, etodolac, indocin, ketorolac, natumetone, sulindac, tolmetin, sulfinpyrazone, dipyridamole, ticlopidine, valdecoxib, rofecoxib, piroxicam, meloxicam, meclofenamate sodium, mefenamic, cyclophosphamide, cyclosporine micromulsion, chlorambucil, anagrelide, clopidogrel, and cilostazol, where the concentration of the anti-inflammatory agent is in the range of 10 to 200 mg/liter. Following soaking in the third solution, the tissue is further soaked in a solution of alcohol, water and an anti-thrombic agent which may be selected from the group consisting of heparin, ardeparin, enoxaparin, tinzaparin, danapariod, lepiruden and hirudin. The concentration of the anti-thrombic agent may be in the range of 100 to 1,000 IU/ml.

In an alternative method, after washing, the tissue may be soaked in a solution of alcohol, water, an anti-inflammation agent, and an anti-thrombic agent having a concentration in the range of 100 to 1,000 IU per ml. The anti-inflammatory agent and anti-thrombic agent in the alternative method are selected from the same groups identified above.

In another variant for non-cardiovascular applications such as orthopedics, neurological, and urological applications, after washing, the tissue is soaked in an anti-inflammatory solution containing alcohol, water and an anti-inflammatory agent selected from the group consisting of indomethacin, ibuprofin, aspirin, choline salicylate, difunisal, magnesium salicylate, magnesium choline salicylate, salsalate, flurbiprofen, fenoprofen, ketoprofen, naprosen, naproxen sodium, oxaprozin, diclofenac sodium, diclofenac misoprostol, etodolac, indocin, ketorolac, natumetone, sulindac, tolmetin, sulfinpyrazone, dipyridamole, ticlopidine, valdecoxib, rofecoxib, piroxicam, meloxicam, meclofenamate sodium, mefenamic, cyclophosphamide, cyclosporine micromulsion, chlorambucil, anagrelide, clopidogrel, and cilostazol.

Further details of the membrane processing procedure are described in U.S. Pat. Nos. 7,008,763; 7,736,845 and 7,687,230, the entire disclosures of each of which are incorporated herein by reference.

A particularly preferred natural membrane for use in the present invention is pericardium. Pericardium is a biological three-layered membrane routinely used in medical procedures such as dura replacement in cranial surgery, glaucoma implant surgery and tracheal reconstruction. Pericardium is a natural barrier membrane and will naturally occlude cells. Pericardium's cell occlusiveness allows the underlying defect space and osseous graft to be predictably isolated preventing soft tissue ingrowth while allowing nutrient passage to occur during healing. Pericardium may be used in dental procedures where resorbable barrier membranes are currently used.

The pericardium is a thin membrane that surrounds the heart. It is also called the “heart sack” in laymen term. It can be taken from bovine, porcine, equine or other large animals. The tissue is mechanically strong but very pliable or flexible. The predominant tissue matrix component is type I collagen in an adult animal, but it also contains many different minor collagen types as well as all the other extra-cellular matrix components that exist in other connective tissues in different layers. The tissue is naturally covered with endothelial-like lining cells on one side. There are other connective tissue cells inside the different layers of the tissue. Blood vessels can also be found inside the tissue. Although the type I collagen component of the tissue is very similar between pericardial tissues from different mammalian sources, the non-type I collagen components are not compatible for cross-species implantation. Hence, it is necessary to process the membrane to prevent inflammation and/or tissue rejection before it can be implanted into a patient.

A containment member suitable for spinal surgery (vertebra augmentation) may be constructed as follows. The container is comprised of two halves. Each half is a cylinder with a round bottom and an open end. One of the halves can be inserted into the other to form a closed capsule. Optionally water-soluble windows may be provided at desired locations. The length of the closed container can be adjusted by compressing or pulling the two halves against each other. Optionally, the halves may have an other than round cross-sectional configuration, i.e., an oval, uneven or irregular shape. The walls of the containment member are strong enough to contain bone graft material to prevent particles thereof from falling out of the containment member. The containment member can be configured with any three dimensional shape that fits into a space where regeneration of bone is desired. The shape can be further manipulated to the desired 3-D structure after the containment member is filled with bone graft material.

Although the containment member may be provided one or more windows, the window material prevents passage of bone augmentation material out of the containment member before implantation. The windows can be clearly identified so that the container can be oriented properly during placement at the surgical site. After implantation, the material that forms the window is dissolved quickly by the body fluid to allow the bone graft material to be in direct contact with tissues where progenitor bone cells to enter the container and form bone. The rest of the material may be resorbed slowly or not resorbed at all.

The material of the non-resorbed portion of the container consists of intact collagen fibers, thin intact tissue membranes or other biocompatible material such as natural bio-polymers and synthetic polymers. The intact tissue membranes or bio-polymers are derived from natural sources but treated to reduce or eliminate rejection by the recipient. The treatments can be chemical modification, extraction or digestion to remove antigenic determinants, or bonding of a biocompatible polymer to mask the antigenic determinants. The window material that disappears after implantation consists of denatured collagen, gelatin, other water soluble biomaterials or synthetic polymers that is strong when dry but dissolves in saline. The window material becomes flexible when wet but maintains enough strength for manipulation at room temperature for at least 30 min. The rest of the container also becomes flexible but is much stronger so it can maintain the manipulated shape and integrity in order for the implant to be compressed and placed into the desired surgical site.

Multiple windows can be provided at different location on the containment members so that progenitor cells from different tissue sites of the surgical location can enter the container. The container has the ability to direct progenitor cells to enter the container through the windows after the soluble material is dissolved but slows other non-desirable cell types to enter because the non-water-soluble portion of the container (spine capsule) is less porous.

For orthopedic applications, the containment member may be of any shape or form. A container that can be shaped to fit and match an existing device, design or shape so that it can be a near perfect fit next to the device, design or shape. The surface(s) as well as the thickness throughout that fits the other device, design or shape is made of denatured collagen, gelatin, or other water soluble biomaterials or synthetic polymers. The rest of the surface(s) may be made of intact collagen fibers or thin intact tissue membranes, or synthetic polymers as in the spine capsule described above that is non-water-soluble and remains strong when wet. If desired, multiple windows of water-soluble material can be disposed on the non-water-soluble membrane portion of the containment member. The non-water-soluble portion of the containment member is strong when wet. The non-water-soluble portion may be resorbed in periods of weeks, months, years or not resorbed at all. The non-water-soluble portion also can be strong enough for “fixation” to bone or hard surfaces by tags or screws. If desired, the containers may be provided with outwardly extending flaps which serve as points of attachment for fixing the containment member in a desired implanted location.

The present invention also includes a method of augmenting a bone comprising the steps of surgically accessing the bone to be augmented, providing a containment member made from a natural membrane isolated from an animal donor and processed to avoid inflammation or tissue rejection; disposing said containment member packed with bone augmentation material proximate the bone to be augmented, and securing the containment member in position. The containment member may first be packed with bone augmentation material and then disposed and secured in the desired position. Alternatively, the containment member can first be disposed and secured in the desired location and then packed with bone augmentation material. The securing may be effected by suturing, by using bone tacks and/or by simply closing an incision over the implanted containment member.

### BRIEF DESCRIPTION OF THE DRAWINGS

The invention will be described in further detail hereinafter with reference to a illustrative embodiments shown in the accompanying drawing figures, in which:

FIG. 1 shows a implantable containment device according to the present invention intended particularly for orthopedic bone augmentation surgery, especially spine surgery, in an open state:

FIG. 2 shows the implantable containment device of FIG. 1 in a closed state; and

FIG. 3 is a representation of a curved containment device particularly suited for oral surgery bone augmentation operations such as alveolar ridge augmentation.

It should be understood that these depictions are only intended as illustrative examples and that the containment member of the invention may exist in a variety of configurations other than those shown in the drawings.

### DETAILED DESCRIPTION

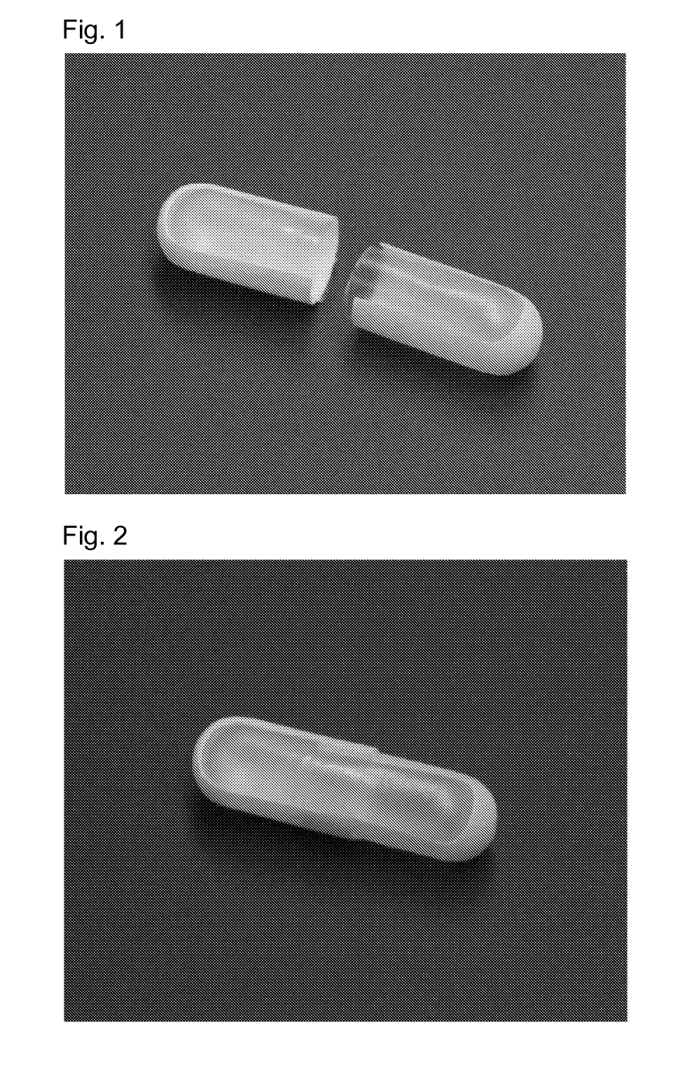

FIGS. 1 and 2 show a containment member according to the present invention particularly adapted for orthopedic applications. The containment member comprises a pair of tubular sections each having a closed end and an open end. The open ends are sized and configured to mate with each other so that the open end of one section can be inserted into the open end of the other section to form a closed capsule. FIG. 1 shows the sections separated from each other, and Fig. shows the sections joined to form a closed containment member. By varying the depth of insertion of the one section into the other, the length of the containment member can be adjusted as desired to fit differing surgical sites.

One or more areas of the containment member may be provided with a “window” of water soluble gelatinous material which rapidly dissipates upon exposure to bodily fluids after implantation. The window is designed to be disposed proximate the bone to be augmented so that upon dissipation of the window, the bone augmentation material contained within the capsule is directly exposed to the bone to be augmented while the remainder of the capsule serves to retain the bone augmentation material in the desired location and maintain the bone augmentation material in the desired configuration.

The embodiment shown in FIGS. 1 and 2 has a window extending substantially along the entire length of one side of each section of the capsule so that the bone graft material contained within the capsule can be exposed to an adjacent bone along the entire length of the capsule, but it will be readily appreciated by persons skilled in the art that one or more windows could be formed in one or both of the capsule sections to achieve a desired degree of contact between the bone graft material and the bone to be augmented.

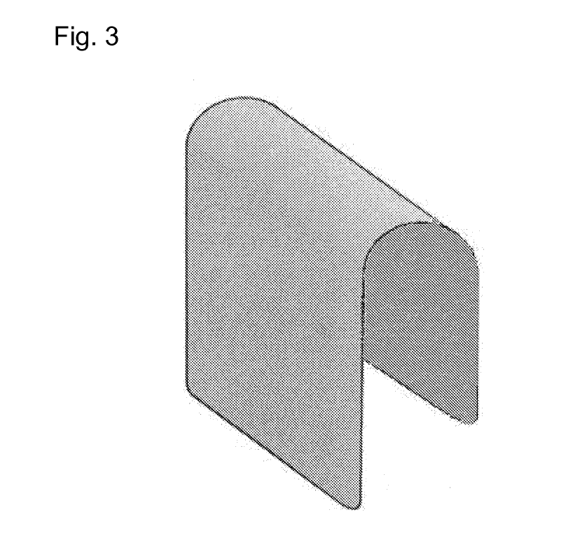

FIG. 3 shows another preferred embodiment of the containment member of the invention. This embodiment is particularly designed for posterior mandible applications in dental surgery. The posterior mandible containment member takes the form of a sheet bent into a bight with two leg sections of uneven length joined by a curved center section. This design allows the containment member to be placed securely over the mandible, after which it can be securely tacked in place by placing bone tacks through one or both legs. The uneven lengths of the two leg sections allow the containment member to better fit the typical dimensions of the oral cavity adjacent the posterior mandible. The optimum dimensions of the containment member will necessarily vary depending on the size of the patient in whom the containment member is to be employed. However, in general the containment member may advantageously have an overall length of 35±5 mm; the two legs may have heights of 25±5 mm and 16±3 mm, respectively; and the spacing between the legs (i.e. the diameter of the curved section joining the two legs) may be about 8±1 mm. Moreover, the processed natural membrane material, from which the containment member is formed, can readily be trimmed to fit by the surgeon upon implantation. Then the tunnel or chamber formed under the curved center section between the containment member and the mandible can be filled as needed with bone augmentation material.

In alveolar ridge augmentation of atrophied jawbones to provide sufficient bone depth to facilitate stable implantation of a dental implant, a principal difficulty is the maintenance of the desired ridge shape, both as to height and as to width. Containment members having arcuate configuration depicted in FIG. 3 have been found to be highly advantageous for alveolar ridge augmentation, especially vertical alveolar ridge augmentation. The containment member of FIG. 3 has a number of important advantages for guided tissue regeneration. It can be readily produced in lengths sufficient to contain a relatively long bone graft and can be readily trimmed to a desired length for shorter bone grafts. Thus, it is unnecessary to manufacture and maintain an inventory of different sized containment members for bone grafts of different lengths because a single standard size can be readily adapted to differing size requirements.

As previously noted, in order to render non-human tissues, such as pericardium, implantable in a human patient, the tissue must be processed to prevent the occurrence of inflammation and or tissue rejection by a chemical process such as the processes disclosed in U.S. Pat. Nos. 7,008,763; 7,736,845 and 7,687,230. The chemical process oxidizes and removes extractable non-collagen components without using an enzyme digestion process which weakens and partially denature the tissue matrix. The process also masks non-extractable contaminants by bonding polyethylene glycol to the surface of the collagen fibers. A small amount of anti-inflammatory agent is incorporated inside the tissue matrix to suppress the initial and immediate post-traumatic inflammatory response to the implanted tissue. After washing the tissue with different solvents, the tissue is essentially non-distinguishable from the original native tissue in its mechanical properties. Biologically, the tissue is accepted by the host with minimal or no foreign body or immune response. When implanted in different sites, appropriate connective cells such as endothelial cells, epithelial cells are found on the surface of the implanted pericardium. Smooth muscle alpha-actin positive cells—presumed to be smooth muscle cells or myofibroblasts, and fibroblasts are found inside the tissue matrix. Therefore, the implanted tissue effectively becomes a template for the host to regenerate it into a living tissue. The effectiveness of such treatments is documented by extensive data and results from animal implant studies as well as human use in cardiovascular (blood vessels and heart valves) and orthopedic (ACL) applications. The foregoing treatment process (L-Hydro process) yields fresh-like tissue which is stored in liquid phase. To improve its handling properties, the tissues may be subjected to further processing to dry and compact the tissue matrix into a thin membrane with defined form and shape.

The processed natural membrane containment member of the present invention has particular advantages for dental surgery applications over reconstituted collagen membranes. For certain oral surgery applications, the desirable stability of the membrane and specified resorption time is required. Collagen dental implant from bovine tendon lacks the strength when the membrane is thin. For a more stable membrane, the thickness is increased but the thickness of the membrane reduces the available space to pack bone graft material underneath it. Pericardial tissue offers a very thin tissue yet it offers a desirable bio-stability. Collagen dental implant from bovine tendon is a reconstituted membrane of collagen fibers which are disrupted during the purification process. In contrast, the processed pericardial tissue of the present invention is essentially a non-perturbed tissue with minor components removed or masked. The collagen dental implant from bovine tendon swells significantly when rehydrated, but the processed pericardial tissue swells very little when rehydrated. The collagen dental implant from bovine tendon does not have any organizational alignment of collagen fibers, whereas the processed pericardial tissue of the present invention retains all native tissue fiber alignments. Finally, the collagen dental implant from bovine tendon tends to crack with excess bending during surgical implantation. Because it is much thinner with intact native collagen fiber alignments, the processed natural tissue of the present invention will not have major cracks during handling.

Membrane processing to prevent the occurrence of inflammation and tissue rejection upon implantation proceeds generally as follows. Membrane tissues, such as pericardial tissues, from different animal sources are first treated by L-Hydro or other treatment methods and cleaned and precut to approximate sizes needed for the intended application. The tissues are then transferred into a final solvent solution through a series of changes. If desired, the tissues may be compressed and molded into shape in the middle of the solvent solution treatment in one or more of the solutions. Afterwards, the membrane tissues are dried under a controlled and clean environment and then heated. The products are then trimmed to the final shapes and sizes and subjected to a final radiation sterilization process.

The containment members of the invention are intended to be filled with a bone graft material such as an hydroxy apatite, either before or at the time of surgical placement. Numerous such materials are well known in the art and are commercially available from various manufacturers. Because the containment member of the present invention is biocompatible and resorbable, it is unnecessary to perform a second surgery to remove the containment member after the bone graft has achieved a sufficient degree of osseointegration. Instead, the containment member of the invention can simply be left in place until it is naturally absorbed by the patient's body.

Because they are intended for surgical applications, the containment members of the invention are preferably manufactured as sterile products and then distributed in sterile packaging. If desired they can first be packaged and then sterilized by exposure to gamma radiation.

The containment members of the present invention are not be absolutely rigid, but instead exhibit sufficient flexibility that the surgeon can bend or deform it to a desired configuration to fit the surgical installation site without cracking or creasing. After placement by the surgeon, the containment member can be sutured in place or can be held in the desired location by conventional bone tacks or bone screws.

The processed membrane containment members of the present invention have several advantages compared to containment members heretofore in use. Containment members made of reconstituted collagen fibers are molded from a fiber slurry and consequently have no definitive collagen fiber orientation. The processed membrane containment member of the present invention retain the native tissue orientation of the fibers. Containment members made of reconstituted collagen fibers may become rigid or brittle when dry and swell and have limited tensile strength when wet. The processed membrane containment members of the present invention maintain a high degree of flexibility and are significantly more resistant to breakage when dry, and exhibit minimal or no swelling and retain the natural strength of the native tissue when wet. To maintain structural integrity containment members made of reconstituted collagen fibers must have a greater thickness then the processed membrane containment members of the present invention. Moreover, the processed membrane containment members of the present invention may exhibit up to double the stability of containment members made of reconstituted collagen fibers and retain their structural integrity for a period of a year or more. Finally, containment members made of reconstituted collagen fibers must eventually be resorbed for tissue regeneration to occur, whereas the processed membrane containment members of the present invention can be integrated as a living regenerated tissue.

Applications of the processed natural membrane containment members of the present invention include dental applications, spine graft containment, knee surgeries and revisions and other orthopedic applications.

The processed natural membrane containment members of the present invention are particularly suitable for use in dental ridge augmentation procedures similar to those which use reconstituted collagen containment members. The processed natural membrane containment members of the invention provide the surgeon successful results where the use a thinner, more flexible, longer lasting membrane is desired. They can be produced in various sizes and additional configurations and can be readily trimmed at the time of implantation to fit a given surgical site. The dental processed natural membrane configurations are designed for augmentation of one, two or three teeth as well as a disc and a unique 3 dimensional sinus repair membrane.

The processed natural membrane containment members of the present invention are also especially suited for use in spinal surgeries. A particularly suitable configuration comprises a pair of mating capsule sections that form a closed pocket with variable (adjustable) lengths. The capsule sections may optionally include a gelatinous window extending all the way to the free edge of the capsules so the open ends are closed together with the closed opposite ends oriented away from each other at opposite ends of the resulting assembly. The material which forms the window is water soluble and should dissipate very rapidly once it is in contact with body fluid. Therefore it will create a window for host bone cells or progenitor cells to migrate in to the pocket for bone induction quickly yet retain the bone graft materials for the longer, desired period of time. The processed membrane containment member does not serve as a structural “fixation” support for the sections of the vertebral column, but instead serves as containment for bone graft material which induces new bone that will fix the sections. The role of the membrane is to maintain the bone graft material, which usually is a clump or a puddle of loose particles, together so it is packed with a predefined compactness dictated by the particle size of the bone graft material. If the particles are not packed together, the space between the particles will be increased too much for an ideal bone induction process.

A specially configured membrane containment member according to the present invention can be used to contain cancellous bone grafts around knee implants. Currently surgeons only pack the bone grafts around the implant and close the wound with the result that the implanted bone graft material is not stable and can shift and/or deform. The containment member of the present invention addresses this issue of bone graft materials not being contained and can be used to help “contain” the bone graft material at suitable locations around the knee implant. In particular, a processed natural membrane containment member according to the present invention can be shape to a desired configuration and then secured in place in a knee joint by suturing or with bone tacks and then subsequently packed with bone graft material. Alternatively, a closed capsule filled with bone graft material and having a window region which dissipates more rapidly than the remainder of the capsule can be surgically implanted with the window region proximate the bone to be augmented.

Various configurations of processed natural membrane containment member can also be used as a container/retainer for bone grafting materials in short bones, long bones, and other orthopedic applications.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A surgically implantable containment member for maintaining a bone augmentation material in a desired location and/or configuration following implantation in a mammalian patient, wherein said containment member is comprised of a natural membrane isolated from an animal donor and processed to avoid inflammation or tissue rejection.

2. A containment member according to claim 1, wherein following the processing said containment member retains the natural membrane structure of the isolated natural membrane.

3. A containment member according to claim 1, wherein the isolated natural membrane is a pericardial membrane.

4. A containment member according to claim 1, wherein said containment member has the form of a cylindrical capsule formed of two sections each of which has a closed end and an open end with the open end of one section sized for mating insertion into the open end of the other section to form a closed containment structure.

5. A containment member according to claim 4, wherein at least one section comprises a window of rapidly resorbable material adapted to be disposed proximate a bone to be augmented, and wherein said window dissipates more quickly than the remaining portions of the containment member to provide direct contact between a bone augmentation material contained in the containment member and the bone to be augmented while the remaining portions of the containment member retain the bone augmentation material in a desired position and/or configuration.

6. A containment member according to claim 1, wherein said containment member comprises an arcuate sheet with parallel leg sections joined at one end by a closed curved section.

7. A containment member according to claim 1, further comprising a flap which serves as a point of attachment for fixing the containment member in a desired implanted location.

8. A method of augmenting a bone comprising:
surgically accessing the bone to be augmented;
providing a containment member made from a natural membrane isolated from an animal donor and processed to avoid inflammation or tissue rejection;
disposing said containment member packed with bone augmentation material proximate the bone to be augmented, and
securing the containment member in position.

9. A method according to claim 8, wherein the containment member is first be packed with bone augmentation material and then disposed and secured in the desired position.

10. A method according to claim 8, wherein the containment member is first disposed and secured in the desired location and then packed with bone augmentation material.

11. A method according to claim 8, wherein the securing is effected by suturing, by using bone tacks or by closing an incision over the implanted containment member.

12. A method according to claim 8, wherein said containment member comprises a window of rapidly dissipating material adapted to be disposed proximate a bone to be augmented, wherein said window dissipates more quickly than the remaining portions of the containment member to provide direct contact between a bone augmentation material contained in the containment member and the bone to be augmented while the remaining portions of the containment member retain the bone augmentation material in a desired position and/or configuration.

13. A method according to claim 8, wherein said bone to be augmented is a jawbone.

14. A method according to claim 8, wherein said bone to be augmented is a vertebra.
